(19) United States
(12) Patent Application Publication
(10) Pub. No.: US 2015/0224159 A1
(43) Pub. Date: Aug. 13, 2015

(54) EXTRACTS OF PERILLA FRUTESCENS SEEDS AND THEIR PHARMACEUTICAL USES

(71) Applicant: Medical and Pharmaceutical Industry Technology and Development Center, New Taipei City (TW)

(72) Inventors: Leah Lo, Taipei City (TW); Win-Chin Chiang, Taipei City (TW); Jui-Ching Chen, New Taipei City (TW); Yu-Min Lin, Taipei City (TW); Chien-Chang Wu, Kaohsiung City (TW); Che-Yi Lin, New Taipei City (TW); Chien-Jen Shih, Yilan County (TW); Yu-Hsuan Lin, Taipei City (TW); Tzu-Chun Chen, New Taipei City (TW); Yu-Hsiang Huang, New Taipei City (TW)

(21) Appl. No.: 14/613,065

(22) Filed: Feb. 3, 2015

Related U.S. Application Data
(63) Continuation of application No. 61/938,920, filed on Feb. 12, 2014.

(51) Int. Cl. A61K 36/535; A61K 31/352; A61K 31/216

(52) U.S. Cl. CPC A61K 36/535; A61K 31/216; A61K 31/352

(57) ABSTRACT

Disclosed is an extract of Perilla frutescens seeds that contains rosmarinic acid, luteolin, and apigenin, the weight ratio between rosmarinic acid, luteolin, and apigenin being 0.1-200:0.1-200:1. Also disclosed is a method for treating a psychiatric disorder using the above-described extract or an extract of Perilla frutescens seeds containing at least an active agent selected from the group consisting of rosmarinic acid, luteolin, and apigenin.

### CROSS-REFERENCE TO RELATED APPLICATIONS

This application is the claims the benefit of U.S. Provisional Application No. 61/938,920, filed on Feb. 12, 2012, the content of which is hereby incorporated by reference in its entirety.

### BACKGROUND OF THE INVENTION

Psychiatric disorder is the leading cause of disability in the United States and other developed countries. See Kessler et al., Archives of General Psychiatry 2005, 62(6), 617-27; and the World Health Organization, the global burden of disease: 2004 update, Table A2, Geneva, Switzerland, 2008.

For example, attention deficit hyperactivity disorder (ADHD) is a psychiatric disorder that affects about 6 to 7 percent of children, resulting in poor school performance. See Willcutt, Neurotherapeutics 2012, 9, 490-99. Psychiatric disorders commonly associated with ADHD include oppositional defiant disorder, conduct disorder, developmental coordination disorder, primary disorder of vigilance, bipolar disorder, major depressive disorder, anxiety disorder, obsessive-compulsive disorder, substance use disorder, and restless legs syndrome.

ADHD and associated psychiatric disorders can be treated with stimulants such as methylphenidate and amphetamine. For safety reasons, medications are not recommended for pre-school children. Side effects include decreased appetite, sleep problems, anxiety, irritability, stomachache, headache, slow growth rate, and cardiovascular problems. See National Institute of Mental Health, Attention Deficit Hyperactivity Disorder (ADHD), NIH Publication No. 12-3572 (2012).

There is a need to develop a safe medication for treating ADHD and associated psychiatric disorders.

### SUMMARY OF THE INVENTION

This invention is based on an unexpected discovery that certain extracts of a traditional Asian food, i.e., Perilla frutescens seeds, are useful in treating psychiatric disorders including ADHD.

Thus, one aspect of this invention relates to an extract of Perilla frutescens seeds containing rosmarinic acid, luteolin, and apigenin for treating a psychiatric disorder (e.g., ADHD, oppositional defiant disorder, conduct disorder, developmental coordination disorder, primary disorder of vigilance, bipolar disorder, major depressive disorder, anxiety disorders, obsessive-compulsive disorder, substance use disorders, and restless legs syndrome), in which the weight ratio between rosmarinic acid, luteolin, and apigenin is 0.1-200:0.1-200:1 (e.g., 0.1-20:0.1-20:1, 0.2-10:0.2-10:1, and 0.5-5:0.5-5:1).

Another aspect of this invention relates to an extract of Perilla frutescens seeds prepared by an extracting process including the steps of: (i) providing Perilla frutescens seeds; (ii) mixing the Perilla frutescens seeds and a first solvent at 15 to 80° C. (e.g., 50 to 80° C. and 15 to 50° C.) for 1 to 10 hours to obtain defatted Perilla frutescens seeds; and (iii) mixing the defatted Perilla frutescens seeds and a second solvent at 50 to 100° C. (e.g., 70 to 100° C.) for 1 to 4 hours to obtain the extract of Perilla frutescens seeds that contains rosmarinic acid, luteolin, and apigenin at an amount effective to treat a psychiatric disorder. Of note, the weight ratio between the Perilla frutescens seeds and the first solvent is 1:3-30 and the weight ratio between the Perilla frutescens seeds and the second solvent is 1:5-30.

Examples of the first solvent include, but are not limited to, CO₂, C₆-C₈ alkane, C₂-C₂₀ ether, benzene, C₂-C₂₀ ester, and a combination thereof. The second solvent is typically C₁-C₄ alcohol, C₃-C₁₀ ketone, acetonitrile, ethyl acetate, water, or a combination thereof. When ethanol-water is used, the solvent has an ethanol content of 60% or less by volume (e.g., 10-60% and 45-55%), or 90% or greater by volume (e.g., 90-99% and 93-98%).

Also within the scope of this invention is use of the extract of Perilla frutescens seeds described above for treating a psychiatric disorder or for manufacturing a medicament in the above-mentioned treatment.

Still within the scope of this invention is a method of treating a psychiatric disorder by administering to a subject suffering from a psychiatric disorder an effective amount of an extract of Perilla frutescens seeds containing one or more of rosmarinic acid, luteolin, and apigenin.

The term “extract” refers to a solid, a liquid dispersion, or a solution containing one or more active compounds that are obtained from a plant or a portion thereof. Typically, it can be directly administered to a subject in need thereof or can be readily formulated to afford a pharmaceutical composition.

The term “alkane” refers to a saturated, linear or branched, cyclic or acyclic hydrocarbon. Examples include pentane, isopentane, n-pentane, cyclopentane, n-hexane, 2-methylpentane, 3-methylpentane, 2,3-dimethylbutane, 2,2-dimethylbutane, cyclohexane, heptane, cycloheptane, octane, cyclooctane, and a combination thereof.

The term “ether” refers to an organic compound having the formula ROR′, in which each of R and R′, independently, is an alkyl or aryl group. Examples include diethyl ether, phenyl ether, methyl phenyl ether, and methyl t-butyl ether. The term “alkyl” refers to an alkane moiety missing a hydrogen atom. The term “aryl” refers to hydrocarbon moiety having one or more aromatic rings, which can contain a heteroatom (e.g., N, O, and S). Examples of aryl moieties include phenyl, naphthyl, naphthylene, pyrenyl, anthryl, phenanthryl, furyl, furylene, fluorenyl, pyrrolyl, thienyl, oxazolyl, imidazolyl, thiazolyl, pyridyl, pyrimidinyl, quinazolinyl, quinolyl, isoquinolyl, and indolyl.

The term “ester” refers to an organic compound having the formula RCO₂R′, in which each of R and R′, independently, is an alkyl or aryl group. Examples include methyl acetate, ethyl acetate, propyl acetate, butyl acetate, methyl formate, ethyl lactate, isopropyl acetate, isobutyl acetate, ethyl propate, and a combination thereof.

The term “alcohol” refers to an organic compound having the formula ROH, in which R is an alkyl or aryl group. Examples include methanol, ethanol, propanol, isopropanol, butanol, isobutanol, tertbutanol, ethylene glycol, and a combination thereof.

The term “ketone” refers to an organic compound having the formula RCOR′, in which each of R and R′, independently, is an alkyl or aryl group. Examples include acetone, cyclohexanone, methyl ethyl ketone, ethyl benzyl ketone, and a combination thereof.

The term “treating” refers to application or administration of an effective amount of an extract to a subject suffering from a psychiatric disorder, with the purpose to cure, remedy, relieve, alleviate, or ameliorate the disease or its symptom(s). “An effective amount” refers to the amount of the extract which is required to confer the desired effect on the subject. Effective amounts vary, as recognized by those skilled in the art, depending on route of administration, excipient usage, and the possibility of co-usage with other therapeutic treatments such as use of other active agents. The extract of this invention described above can be preliminarily screened for their efficacy in treating a psychiatric disorder in a mammal by an in vitro assay. For example, an extract can be administered to an animal (e.g., a mouse model) having a psychiatric disorder and its therapeutic effects are then assessed. Based on the results, an appropriate dosage range and administration route can also be determined.

The details of one or more embodiments of the invention are set forth in the description and the drawings below. Other features, objects, and advantages of the invention will be apparent from the description, the drawings, and also from the claims.

### BRIEF DESCRIPTION OF THE DRAWINGS

The invention description below refers to the accompanying drawings, of which:

FIG. 1 includes ten graphs (1a-1j) showing HPLC chromatograms of the rosmarinic acid/luteolin/apigenin relative contents in extracts of the present invention.

FIG. 2 includes two graphs (2a and 2b) showing data of norepinephrine uptake inhibition studies.

FIG. 3 includes three graphs (3a, 3b, and 3c) showing total activities of spontaneously hypertensive rats administered with extracts of the present invention.

FIG. 4 includes three graphs (4a, 4b, and 4c) showing concentrations of norepinephrine, dopamine, and serotonin in dialysates collected from rats administered with an extract of the present invention.

FIG. 5 includes three graphs (5a, 5b, and 5c) showing concentrations of norepinephrine, dopamine, and serotonin in dialysates collected from rats administered with an extract of the present invention.

### DETAILED DESCRIPTION

Perilla frutescens has been cultivated as food in China, Japan, and Korea for more than two thousand years. Its leaves are used in fish stew or with sashimi and its seeds are added to soup for seasoning.

As pointed out above, the extract of Perilla frutescens seeds of this invention can be prepared by mixing the Perilla frutescens seeds and a non-polar solvent (e.g., n-hexane) at 15 to 80° C. for 1 to 10 hours to obtain defatted Perilla frutescens seeds and mixing the defatted Perilla frutescens seeds and a polar solvent (e.g., water and ethanol) at 25 to 100° C. for 1 to 4 hours to obtain a liquid extract that contains rosmarinic acid, luteolin, and apigenin. Optionally, the liquid extract is concentrated and/or further dried to obtain a solid extract of Perilla frutescens seeds. Described below is an exemplary extracting process:

Perilla frutescens dry seeds are defatted by mixing and agitating with n-hexane repeatedly, after which n-hexane is removed from the mixture. The resultant defatted Perilla frutescens seeds are subsequently dried and then mixed with 95% ethanol by volume or water at reflux. Removal of the debris affords a greenish liquid extract of Perilla frutescens seeds.

The extract thus obtained contains at least three active ingredients, i.e., rosmarinic acid, luteolin, and apigenin. Their chemical structures are shown below:

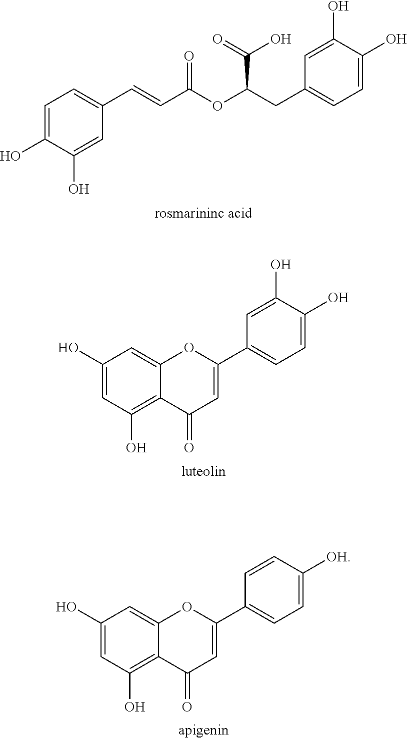 [SMILES: O=C(/C=C/c1ccc(O)c(O)c1)O[C@H](Cc1ccc(O)c(O)c1)C(=O)O ; O=c1cc(-c2ccc(O)c(O)c2)oc2cc(O)cc(O)c12 ; O=c1cc(-c2ccc(O)cc2)oc2cc(O)cc(O)c12]

The liquid extract can be used for treating ADHD and associated psychiatric disorders directly or, if needed, after further concentration or dilution.

Alternatively, the liquid extract is dried to obtain a solid extract of Perilla frutescens seeds. This solid extract can contain 0.1 to 300 mg/g (e.g., 0.1 to 120 mg/g and 0.1 to 60 mg/g) of rosmarinic acid, 0.1 to 250 mg/g (e.g., 0.1 to 100 mg/g and 0.1 to 50 mg/g) of luteolin, and 0.1 to 300 mg/g (e.g., 0.1 to 120 mg/g and 0.1 to 60 mg/g) of apigenin. The total content of rosmarinic acid, luteolin, and apigenin is 0.03 to 67% (e.g., 0.03 to 34% and 0.03 to 17%) by weight of the solid extract. Typically, the fat content is less than 40 wt % (e.g., <30 wt %, <25 wt %, and <20 wt %), the carbohydrate is content is less than 40 wt % (e.g., <35 wt %, <30 wt %, and <25 wt %), and the protein content is less than 15 wt % (e.g., <10 wt % and <5 wt %).

Both the liquid extract and the solid extract can be formulated to afford a pharmaceutical composition for oral administration. Suitable dosage forms include capsules, tablets, emulsions, aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. Oral solid dosage forms can be prepared by amorphous spray dried techniques, hot melt extrusion strategy, micronization, and nano-milling technologies.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of the extract of this invention. Examples of other carriers include colloidal silicon oxide, magnesium stearate, cellulose, sodium lauryl sulfate, and D&C Yellow #10.

One can also use chromatography to enrich active compounds that are effective in treating ADHD and associated psychiatric disorders. Chromatography technologies include paper chromatography, thin layer chromatography, column chromatography (e.g., Diaion HP-20 silica gel column Sephadex cross-linked dextran gel column), gas chromatography, and liquid chromatography (e.g., HPLC). Suitable eluent solvents include water, methanol, acetonitrile, and a mixture thereof. A gradient eluent system can be used. Alternatively, one can also use recrystallization to enrich one or more active components. The recrystallization solvent can be an inorganic or organic solvent, e.g., a solvent in which the desired product has a low solubility at a low temperature, but has a higher solubility at a high temperature. It can also be a solvent pair or a mixture. An even more efficacious product can be thus obtained.

The contents of active compounds can be determined using chromatography or other instruments, such as UV, MS or NMR.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLES 1-10

Ten extracts of Perilla frutescens seeds were prepared.

In Example 1, an extract of Perilla frutescens seeds, i.e., 2396-PF10, was prepared following the procedures described below.

Perilla frutescens dry seeds (100 g) were defatted by mixing and agitating with n-hexane (600 g) at 25° C. for 2 hours, after which n-hexane was removed from the mixture. This n-hexane defatting process was repeated for three more times. The resultant defatted Perilla frutescens seeds were dried in an oven at 60° C. for 24 hours and subsequently mixed with ethanol (95%; 1000 g) in a flask, which was heated and kept at reflux for 1 hour. After cooling to ambient temperature, the mixture was filtered to obtain an ethanol solution. The solid seeds were returned to the flask. Another 1000 g of ethanol was then added to the flask to repeat this extraction process. After the extraction, a second ethanol solution was collected. The two ethanol solutions were combined and dried to obtain 2.89 g of a solid extract of Perilla frutescens, i.e., 2396-PF10. The solid extract was subjected to HPLC analysis described in Example 11 below.

In Example 2, another extract of Perilla frutescens seeds, i.e., 2396-PF10-Hot, was prepared following a similar procedure except that the n-hexane defatting process was conducted at 70° C. for 1 hour twice instead of 25° C. for 2 hours, four times. Extract 2396-PF10-Hot was obtained at a yield of 2.48 g, which was also analyzed by HPLC. See Example 11 below.

In Examples 3-7, five more extracts, i.e., 2396-PF3, 2396-PF5, 2396-PF7, and 2396-PF12, were all prepared following the same procedure used to prepare extract 2396-PF10. The yields were 3.03%, 2.75%, 2.41%, and 2.9%, respectively. These five extracts were analyzed by HPLC. See Example 11. Inhibition of ADHD by 2396-PF3 and 2396-PF7 was evaluated as described in Example 12 below.

In Example 8, Extract PF7-hex-H₂O was prepared following a procedure similar to that for preparing extract 2396-PF10 described above. More specifically, Perilla frutescens dry seeds (800 g) were defatted by mixing and agitating with n-hexane (4800 g) at 25° C. for 2 hours, after which n-hexane was removed from the mixture. This n-hexane defatting process was repeated for three more times. The resultant defatted Perilla frutescens seeds were dried in an oven at 60° C. for 24 hours and subsequently mixed with water (8000 g) in a flask at reflux for 1 hour. The aqueous solution was collected by filtration. The seeds were returned to the flask, to which another 8000 g of water was added to repeat the extraction process. After the extraction, a second aqueous solution was collected. The two aqueous solutions were combined and dried to obtain 49.57 g (a yield of 6.2%) of a solid extract of Perilla frutescens, i.e., PF7-hex-H₂O. This extract was subjected to HPLC analysis described in Example 11 below.

In Example 9, Extract PF7-hex-50EtOH was prepared following a procedure similar to that for preparing extract 2396-PF10. More specifically, Perilla frutescens dry seeds (600 g) were defatted by mixing and agitating with n-hexane (3600 g) at 25° C. for 2 hours, after which n-hexane was removed from the mixture. This n-hexane defatting process was repeated for three more times. The resultant defatted Perilla frutescens seeds were dried in an oven at 60° C. for 24 hours and subsequently mixed with an ethanol-water mixed solvent (ethanol 50% v/v; 6000 g) in a flask at reflux for 1 hour. A solution was collected by filtration. The seeds were returned to the flask, to which another 6000 g of ethanol-water was added to repeat the extraction process. After the extraction, a second solution was collected. The two solutions were combined and dried to obtain 27.05 g (a yield of 4.5%) of a solid extract of Perilla frutescens, i.e., PF7-hex-50EtOH, which was subjected to HPLC analysis described in Example 11 below.

In Example 10, Extract PF7-hex-EA was prepared following a procedure similar to that for preparing extract 2396-PF10. More specifically, Perilla frutescens dry seeds (2400 g) were defatted by mixing and agitating with n-hexane (14400 g) at 25° C. for 2 hours, after which n-hexane was removed from the mixture. This n-hexane defatting process was repeated for three more times. The resultant defatted Perilla frutescens seeds were dried in an oven at 60° C. for 24 hours and subsequently mixed with ethyl acetate (24000 g) in a flask at reflux for 1 hour. An ethyl acetate solution was collected by filtration. The seeds were returned to the flask, to which another 24000 g of ethyl acetate was added to repeat the extraction process. After the extraction, a second ethyl acetate solution was collected. The two ethyl acetate solutions were combined and dried to obtain 14.32 g (a yield of 0.6%) of a solid extract of Perilla frutescens, i.e., PF7-hex-EA. This extract was subjected to HPLC analysis described in Example 11 below.

### EXAMPLE 11

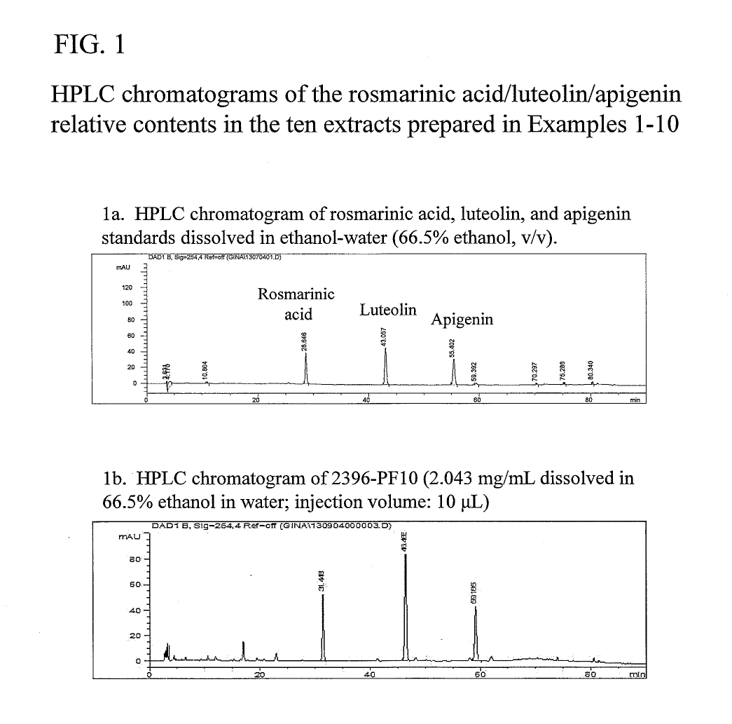

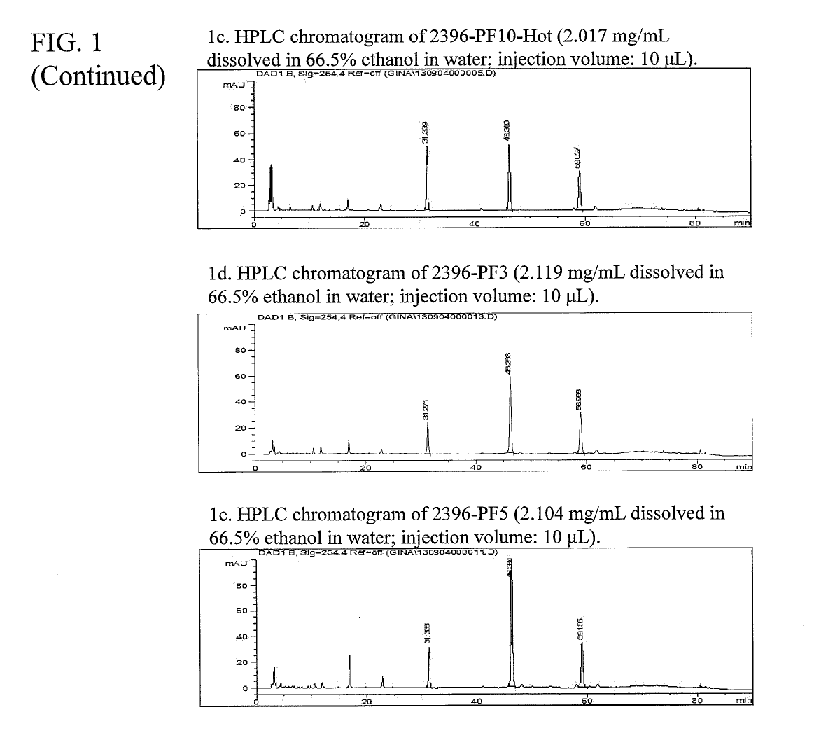

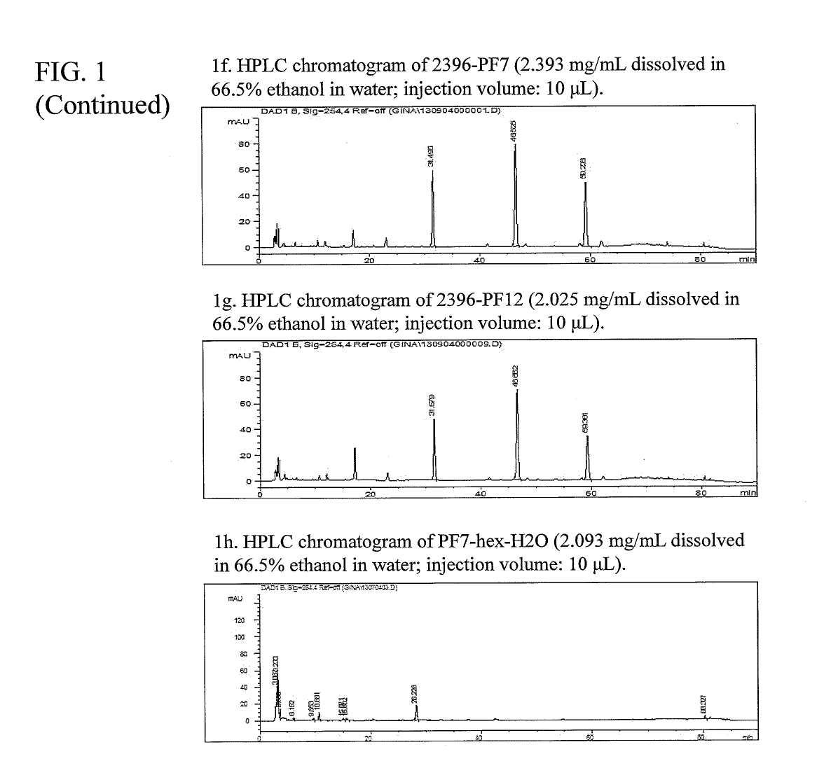

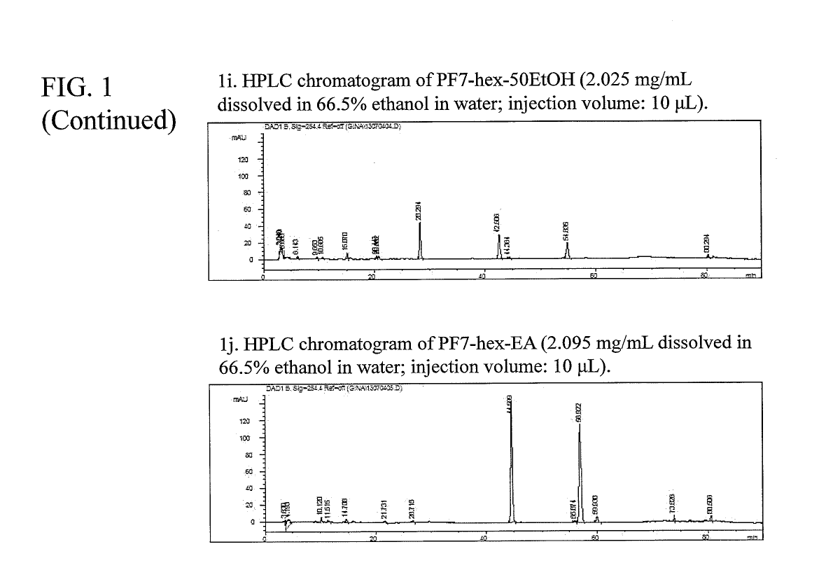

The extracts prepared in Examples 1-10 were analyzed using HPLC. See FIG. 1. The HPLC conditions are described below.

Extracts 2396-PF10, 2396-PF10-Hot, 2396-PF3, 2396-PF5, 2396-PF7, 2396-PF12, PF7-hex-H2O, PF7-hex-50EtOH, and PF7-hex-EA were analyzed by gradient reversed phase HPLC with UV detection at 254 nm. The separation was accomplished using gradient elution with Mobile Phase A (0.1% formic acid aqueous solution) and Mobile Phase B (acetonitrile) on an Atlantis® T3 column (Waters, 5 μm, 4.6×250 mm) at a flow rate of 1.0 mL/min and at 35° C. See Table 1 below for the compositions of the two mobile phases over time.

TABLE 1
HPLC gradient elution

| | Mobile Phase solution | Mobile Phase |
|---|---|---|
| Time | A (0.1% formic acid | solution B |
| (min) | aqueous solution) (%) | (acetonitrile) (%) |
| 0 | 85 | 15 |
| 20 | 78 | 22 |
| 60 | 67 | 33 |
| 80 | 0 | 100 |
| 90 | 0 | 100 |
| 100 | 85 | 15 |

Table 2 below shows the contents of each of rosmarinic acid, luteolin, and apigenin in four extracts, i.e., 2396-PF7, PF7-hex-H₂O, PF7-hex-50EtOH, and PF7-hex-EA. The combined contents of these three ingredients are also shown in this table.

TABLE 2
Rosmarinic acid, luteolin, and apigenin contents in four extracts

| | Rosmarinic | Luteolin | Apigenin | Content |
|---|---|---|---|---|
| | acid (mg/g) | (mg/g) | (mg/g) | (mg/g) |
| 2396-PF7 | 42.58 | 24.75 | 23.04 | 90.37 |
| PF7-hex-H2O | 15.32 | 1.26 | 0.81 | 17.39 |
| PF7-hex-50EtOH | 36.26 | 10.47 | 9.97 | 56.71 |
| PF7-hex-EA | 2.18 | 50.36 | 59.02 | 111.56 |

Table 3 below shows the contents of each of rosmarinic acid, luteolin, and apigenin in six extracts, i.e., 2396-PF3, 2396-PF5, 2396-PF7, 2396-PF10, 2396-PF10-Hot, and 2396-PF12. The combined contents of these three ingredients are also shown in the last column of this table. The ratios between Rosmarinic acid, Luteolin, and Apigenin are shown in Table 4 below.

In 2396-PF7 and 2396-PF10, the fat contents, the carbohydrate contents, and the protein contents were also measured following a procedure described in Chinese National Standards 5035 and 5036 (Taiwan). Extract 2397-PF7 contained fat 26.32 wt %, and extract 2397-PF10 contained fat 25.55 wt %, carbohydrate 31.15 wt %, and protein 7.97 wt %.

TABLE 3
The contents of Rosmarinic acid, Luteolin,
and Apigenin in six extracts

| | Rosmarinic | Luteolin | Apigenin | Content |
|---|---|---|---|---|
| | acid (mg/g) | (mg/g) | (mg/g) | (mg/g) |
| 2396-PF10 | 42.56 | 32.72 | 26.11 | 101.39 |
| 2396-PF10-Hot | 41.12 | 19.23 | 18.08 | 78.43 |
| 2396-PF12 | 39.13 | 27.59 | 21.56 | 88.28 |
| 2396-PF7 | 41.05 | 25.99 | 25.50 | 92.54 |
| 2396-PF5 | 25.28 | 39.63 | 21.74 | 86.65 |
| 2396-PF3 | 18.94 | 22.99 | 18.88 | 60.81 |

TABLE 4
The ratios between Rosmarinic acid, Luteolin,
and Apigenin in six extracts

| | Rosmarinic | Rosmarinic | Luteo- |
|---|---|---|---|
| | acid:Luteolin:Apigenin | acid:Apigenin | lin:Apigenin |
| 2396-PF10 | 1.6:1.3:1 | 1.63:1 | 1.25:1 |
| 2396-PF10-Hot | 2.3:1.1:1 | 2.27:1 | 1.06:1 |
| 2396-PF12 | 1.8:1.3:1 | 1.81:1 | 1.28:1 |
| 2396-PF7 | 1.6:1:1 | 1.61:1 | 1.02:1 |
| 2396-PF5 | 1.2:1.8:1 | 1.16:1 | 1.82:1 |
| 2396-PF3 | 1:1.2:1 | 1.00:1 | 1.22:1 |

### EXAMPLE 12

Extract 2396-PF10 was tested in inhibiting norepinephrine uptake.

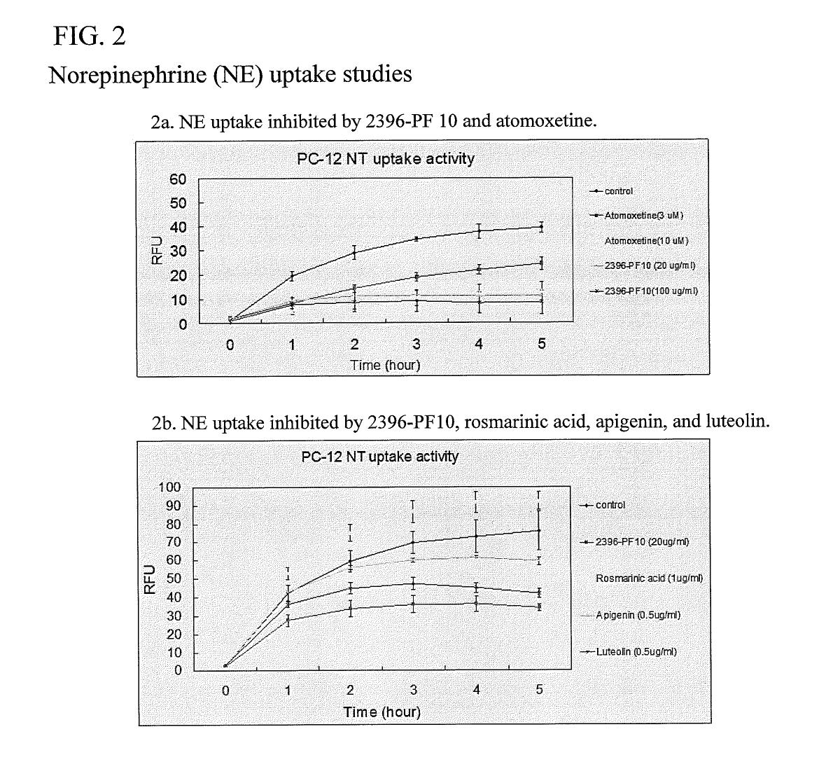

More specifically, PC-12 cells, containing a large amount of norepinephrine (NE) transporters, were seeded in 24-well or 96-well plates at a density of 60,000 or 120,000 cells per well in growth medium. After 24 hours of culture at 37° C. in an atmosphere containing 5% CO₂, the culture medium was removed. Wells were incubated in a Hank's Balanced Salt Solution (HBSS) containing 0.1% bovine serum albumin (BSA) in the absence or presence of extract 2396-PF10 for 60 minutes at 37° C. Subsequently, a fluorescent dye solution was added to each well and the cells were incubated for 60 more minutes. The fluorescent dye solution was then removed and the well was washed with HBSS. The cells were solubilized by adding 1×Trypsin-EDTA. The cell solution was analyzed with a flow cytometer and quantified with an M5 microplate reader. Relative fluorescence units (RFUs) were recorded and shown in FIG. 2.

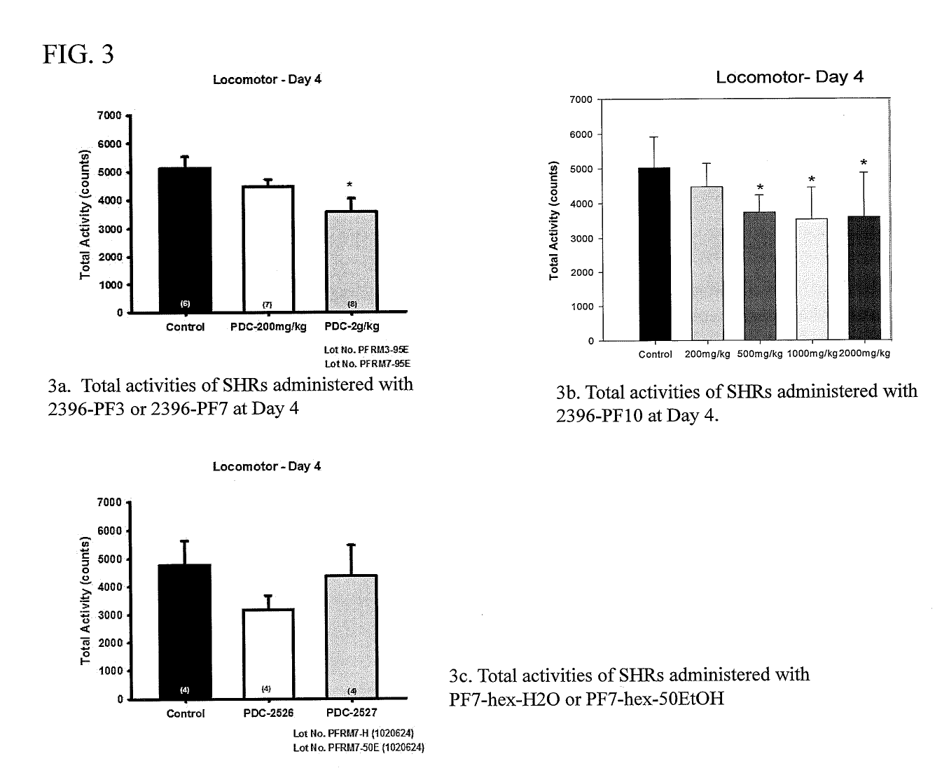

Extract 2396-PF10 unexpectedly inhibited the uptake of NE by PC-12 cells effectively. See FIGS. 2 and 3 for the results as compared to Atomoxetine (a commercial drug for treating ADHD), rosmarinic, luteolin, and apigenin. Note that the data in FIG. 2a were obtained using the density of 60,000 cells per well and the data in FIG. 2b were obtained using the density of 120,000 cells per well.

### EXAMPLE 13

Two extracts, i.e., 2396-PF3 and 2396-PF7, were used to assess efficacy in treating ADHD following the procedure described below. See also Sagvolden et al., Biological Psychiatry 2005, 57, 1239-47; Sagvolden, Neuroscience Biobehavioral Review 2000, 24, 31-39; Wiersema et al., Journal of Neural Transmission 2005, 1417-30; and Okamoto, Japanese Circulation Journal 1963, 27, 282-93.

Spontaneously hypertensive rats (SHRs) were used as ADHD models. Their locomotor activity was assessed using an automated locomotor activity analysis system (4 channels, San Diego Instruments). More specifically, an SHR was placed in a closed cage (40 cm×40 cm) having a grid of infrared beams mounted horizontally every 2.5 cm. Spontaneous locomotor activities were recorded. Total movements were counted every five minutes during a one-hour period. All assays were conducted between 9:00 am and 10:00 am in a quiet environment. For a more detailed description of the procedure, see also Furuie et al., Behavioural Pharmacology 2013, 24, 678-83; Hiraide et al., Pharmacology Biochemistry and Behavior 2013, 105, 89-97; van den Bergh et al., Pharmacology Biochemistry and Behavior 2006, 83, 380-90; and Yang, et al., Brain Research Bulletin 2006, 71, 301-10.

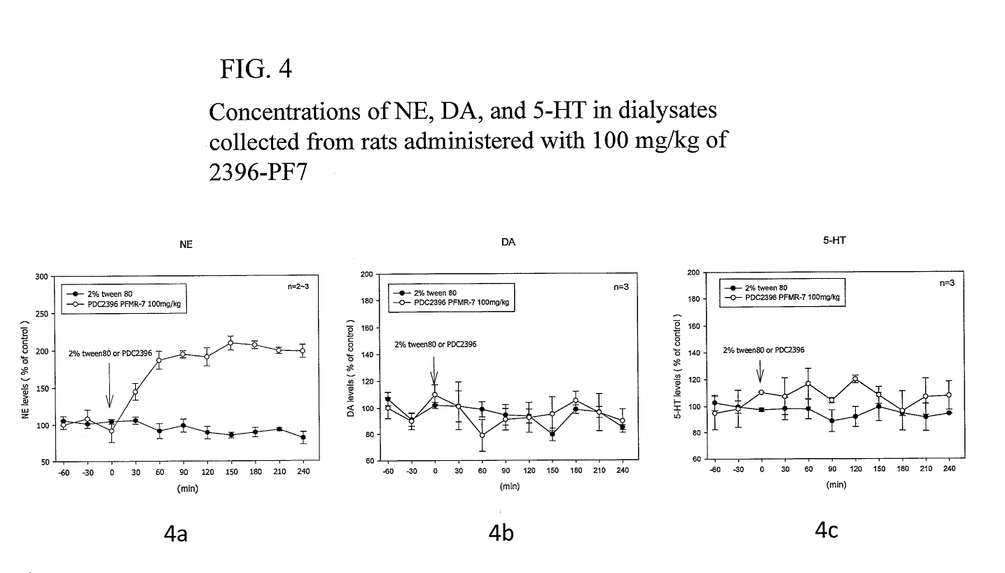

Either 2396-PF3 or 2396-PF7 was administered to a group of SHRs (n=7 or 8) orally at a dose of 200 mg/kg or 2000 mg/kg. Saline, instead of 2396-PF3 and 2396-PF7, was used in a control group (n=6). In each SHR administered at a dose of 200 mg/kg or 2000 mg/kg (either 2396-PF3 or 2396-PF7), movements were counted at Day 4. The average number of movements (counts) of all SHRs in 1 hour was calculated and shown in FIG. 3a. Unexpectedly, 2396-PF3 and 2396-PF7 significantly inhibited SHR hyperactivities at Day 4 in both the group administered at a dose of 2000 mg/kg. See FIG. 4. Note that, in this figure, Lot No. PF RM3-95E refers to 2396-PF3, Lot No. PF RM7-95E refers to 2396-PF7, and PDC refers to both 2396-PF3 and 2396-PF7.

Extract 2396-PF10 was used to assess efficacy in treating ADHD at different dosages (200 mg/kg, 500 mg/kg, 1000 mg/kg, and 2000 mg/kg) following the procedure described above. Saline was used in a control group. The results are shown in FIG. 3b.

Unexpectedly, 2396-PF10 effectively inhibited SHR hyperactivities at a dose as low as 500 mg/kg.

PF7-hex-H₂O and PF7-hex-50EtOH, both at a dose of 2000 mg/kg, were also used to assess efficacy in treating ADHD following the procedure described above. Saline was used in the control group. The results are shown in FIG. 3c. Note that, in this figure, both PDC-2526 and PFRM7-H refer to PF7-hex-H₂O and both PDC-2527 and PFRM7-50E refer to PF7-hex-50EtOH.

Unexpectedly, PF7-hex-H₂O effectively inhibited SHR hyperactivities after being administered to test animals for 4 days. On the other hand, PF7-hex-50EtOH did not show inhibition of SHR hyperactivities.

### EXAMPLE 14

Extract 2396-PF7 was used in a brain microdialysis following the procedure set forth below. For a detailed description of the procedure, see Ago et al., Neuropsychopharmacology 2005, 30, 43-51; and Amargos-Bosch et al., Journal of Neurochemistry 2007, 102, 550-61.

More specifically, male Sprague-Dawley rats, test animals, were supplied with unlimited water and diet in a cage having a temperature of 22±1° C. and light from 7:00 am to 7:00 pm. A microdialysis probe was inserted into the brain at the prefrontal cortex area. An artificial extracellular fluid (149 mM NaCl, 1.2 mM CaCl₂, 1.2 mM MgCl₂, and 2.8 mM KCl) was infused at 1 mL/min for 2 hours. Subsequently, dialysates were collected every 20 minutes and analyzed using a HPLC system. The first three dialysates were collected as control samples. The rats were then administered with extract 2396-PF7 at a dose of 100 mg/kg or 300 mg/kg via intraperitoneal injection. More dialysates were collected and analyzed.

Concentrations of norepinephrine (NE), dopamine (DA), and serotonin (5-HT) in the dialysates were determined by a HPLC system. Low levels of NE, DA, and 5-HT are associated with ADHD. See Prince, Journal of Clinical Psychopharmacology 2008, 3, Suppl 2, S39-45.

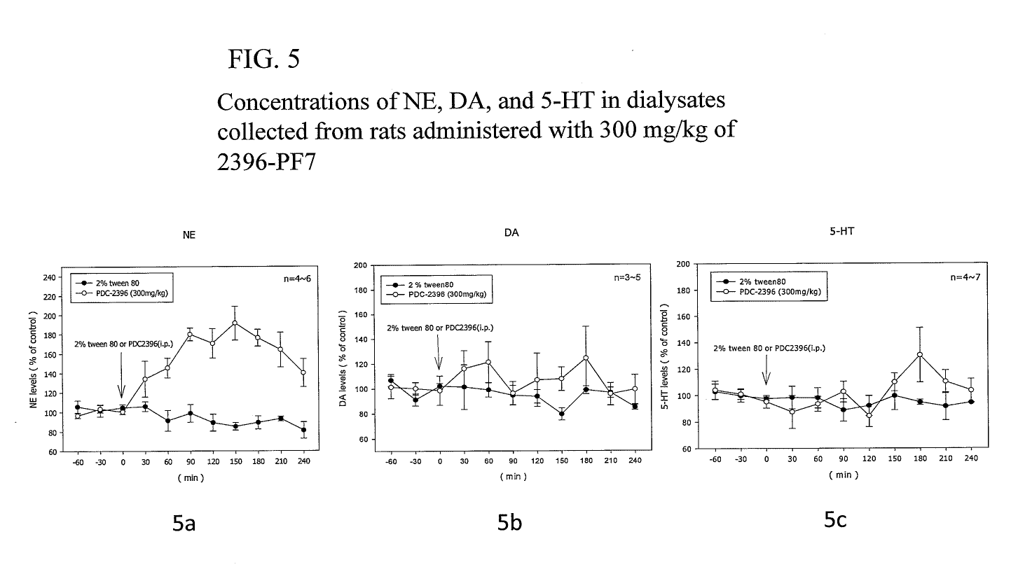

The results were shown in FIGS. 4 and 5. Note that, in both figures, PDC2396 or PDC2396 PFRM-7 refers to 2396-PF7. Tween 80 (2% in aqueous solution), shown in both figures, was used as vehicle.

Unexpectedly, 2396-PF7, at either dose of 100 mg/kg or 300 mg/kg, increased the level of NE in the brains of tested rats.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. An extract of Perilla frutescens seeds comprising rosmarinic acid, luteolin, and apigenin for treating a psychiatric disorder, wherein the weight ratio between rosmarinic acid, luteolin, and apigenin is 0.1-200:0.1-200:1.

2. The extract of claim 1, wherein the weight ratio between rosmarinic acid, luteolin, and apigenin is 0.1-20:0.1-20:1.

3. The extract of claim 2, wherein the weight ratio between rosmarinic acid, luteolin, and apigenin is 0.2-10:0.2-10:1.

4. The extract of claim 4, wherein the weight ratio between rosmarinic acid, luteolin, and apigenin is 0.5-5:0.5-5:1.

5. The extract of claim 1, wherein the psychiatric disorder is attention deficit hyperactivity disorder, oppositional defiant disorder, conduct disorder, developmental coordination disorder, primary disorder of vigilance, bipolar disorder, major depressive disorder, anxiety disorder, obsessive-compulsive disorder, substance use disorder, and restless legs syndrome.

6. The extract of claim 5, wherein the psychiatric disorder is attention deficit hyperactivity disorder.

7. An extract of Perilla frutescens seeds, wherein the extract is obtained by:
providing Perilla frutescens seeds,
mixing the Perilla frutescens seeds and a first solvent at 15 to 80° C. for 1 to 10 hours to obtain defatted Perilla frutescens seeds, and
mixing the defatted Perilla frutescens seeds and a second solvent at 50 to 100° C. for 1 to 4 hours to obtain the extract of Perilla frutescens seeds that contains rosmarinic acid, luteolin, and apigenin at an amount effective to treat a psychiatric disorder, the weight ratio between rosmarinic acid, luteolin, and apigenin being 0.1-200:0.1-200:1, wherein
the first solvent is CO₂, C₆-C₈ alkane, C₂-C₂₀ ether, benzene, C₂-C₂₀ ester, or a combination thereof;
the second solvent is C₁-C₄ alcohol, C₃-C₁₀ ketone, acetonitrile, ethyl acetate, water, or a combination thereof, provided that when ethanol-water is used, ethanol has a content of at most 60% by volume or at least 90% by volume;
the weight ratio between the Perilla frutescens seeds and the first solvent is 1:3-30; and
the weight ratio between the Perilla frutescens seeds and the second solvent is 1:5-30.

8. The extract of claim 7, wherein the first solvent is hexanes and the second solvent is water, ethanol, acetonitrile, acetone, ethyl acetate, or a combination thereof.

9. The extract of claim 8, wherein the Perilla frutescens seeds are mixed with the first solvent at 50 to 80° C., and the defatted Perilla frutescens seeds are mixed with the second solvent at 70 to 100° C.

10. The extract of claim 8, wherein the Perilla frutescens seeds are mixed with the first solvent at 15 to 50° C., and the defatted Perilla frutescens seeds are mixed with the second solvent at 70 to 100° C.

11. The extract of claim 7, wherein the Perilla frutescens seeds are mixed with the first solvent at 50 to 80° C., and the defatted Perilla frutescens seeds are mixed with the second solvent at 70 to 100° C.

12. The extract of claim 7, wherein the Perilla frutescens seeds are mixed with the first solvent at 15 to 50° C., and the defatted Perilla frutescens seeds are mixed with the second solvent at 70 to 100° C.

13. The extract of claim 7, wherein the psychiatric disorder is attention deficit hyperactivity disorder, oppositional defiant disorder, conduct disorder, developmental coordination disorder, primary disorder of vigilance, bipolar disorder, major depressive disorder, anxiety disorder, obsessive-compulsive disorder, substance use disorder, and restless legs syndrome.

14. The extract of claim 13, wherein the psychiatric disorder is attention deficit hyperactivity disorder.

15. A method of treating a psychiatric disorder, the method comprising administering to a subject having the psychiatric disorder an effective amount of an extract of any of claims 1-4 and 7-12.

16. The method of claim 15, wherein the psychiatric disorder is attention deficit hyperactivity disorder, oppositional defiant disorder, conduct disorder, developmental coordination disorder, primary disorder of vigilance, bipolar disorder, major depressive disorder, anxiety disorder, obsessive-compulsive disorder, substance use disorder, and restless legs syndrome.

17. The method of claim 16, wherein the psychiatric disorder is attention deficit hyperactivity disorder.

18. A method of treating a psychiatric disorder, the method comprising administering to a subject having the psychiatric disorder an effective amount of an extract of Perilla frutescens seeds containing at least an active agent selected from the group consisting of rosmarinic acid, luteolin, and apigenin.

19. The method of claim 18, wherein the psychiatric disorder is attention deficit hyperactivity disorder, oppositional defiant disorder, conduct disorder, developmental coordination disorder, primary disorder of vigilance, bipolar disorder, major depressive disorder, anxiety disorder, obsessive-compulsive disorder, substance use disorder, and restless legs syndrome.

20. The method of claim 19, wherein the psychiatric disorder is attention deficit hyperactivity disorder.
